(19) United States
(12) Patent Application Publication
(10) Pub. No.: US 2013/0331736 A1
(43) Pub. Date: Dec. 12, 2013

(54) APPLIED KINESIOLOGY (AK) METHOD AND APPARATUS

(71) Applicant: Fred Sands, Los Angeles, CA (US)

(72) Inventor: Fred Sands, Los Angeles, CA (US)

(21) Appl. No.: 13/494,088

(22) Filed: Jun. 12, 2012

(51) Int. Cl. A61B 5/103

(52) U.S. Cl. 600/595

(57) ABSTRACT

Testing apparatus and methods eliminate uncertainties associated with applied kinesiology (AK) evaluation. The invention comprises a housing and a vertical member extending upwardly from the housing. The member includes an upper platform to receive a pressure-applying body part and a lower portion that enters into the housing wherein it is coupled to a device to measure the applied pressure. The measurement device is in communication with a readout on the housing that indicates the applied pressure. The platform, preferably in the form of a cradle, is coupled to a receptacle to hold a food or item to be evaluated. The patient applies a standard measured resistance, and when the item to be tested is held by the patient, the machine measures the decrease in resistance from the body, thereby accurately quantifying the patient's response to the food or other item being tested.

### FIELD OF THE INVENTION

This invention relates generally to applied kinesiology, or AK, and, in particular, to apparatus and methods that provide consistent standardized results by accurately measuring a patient's resistance throughout an AK evaluation cycle.

### BACKGROUND OF THE INVENTION

Applied kinesiology (AK) is used to evaluate structural, chemical, and mental aspects of human health through muscle testing and conventional diagnostic methods. The theory is that every organ dysfunction is accompanied by a weakness in a specific corresponding muscle in what is termed the “viscerosomatic relationship.” A manual muscle test in AK is conducted by having the patient resist using the target muscle or muscle group while the practitioner applies a force. A smooth response is sometimes referred to as a “strong muscle” and a response that was not appropriate is sometimes called a “weak response.” A common test is the arm-pull-down test, or “Delta test,” where the patient resists as the practitioner exerts a downward force on an extended arm. Proper positioning is critical to ensure that the muscle in question is properly isolated to minimize interference from adjacent muscle groups.

“Nutrient testing” is used to examine the response of various of a patient's muscles to assorted chemicals. Gustatory and olfactory stimulation are said to alter the outcome of a manual muscle test, with previously weak muscles being strengthened by application of the correct nutritional supplement, and previously strong muscles being weakened by exposure to harmful or imbalancing substances or allergens.

Many health care professionals, chiropractors and people who practice AK utilize the technique to test patients for foods that they are allergic to, as well as deficient body organs such as the thyroid, adrenals, diaphragm, ribs out of place, etc. This is accomplished with a manual muscle test wherein the patient holds their arm out horizontally while the doctor or health care professional pushes down on their arm to the point where the arm is ready to collapse. The patient then picks up a target food and returns their arm to the original position. If the patient is allergic to the food, his/her arm will drop and the muscle will become much weaker because the electrical/nervous system of the body rejects it.

Numerous studies have been done utilizing this method and there have been inconsistent results. The results have varied between one doctor and another because there are too many variables, one being how hard the patient is resisting. After a few minutes of testing it is not unusual for the patient's arm to lose some of its strength. Likewise, the practitioner cannot be consistent on how hard he/she pushes down on the patient's arm and after a few minutes of testing, the practitioner may not be pushing down as hard.

What is lacking is consistency—i.e., standardization—and that is why the results vary from doctor-to-doctor, patient-to-patient and test-to-test. There is no true baseline to provide standardization of testing and therefore accurate results. The patient's strength may fluctuate as much as 20% to 30% depending on how he/she is feeling at that moment in time or what his/her stamina is like. Likewise, the doctor may not be pushing down as hard and may not be consistent at all times. Therefore, the results obtained through AK can fluctuate greatly because there is a variable on the part of the doctor applying it and, of course, the patient resisting. This method of AK is considered a yardstick with many variables. Therefore, it often cannot be counted upon.

There are devices that measure forces associated with manual muscle testing. U.S. Pat. No. 4,501,148, entitled “Manual Muscle Tester,” the entire content of which is incorporated herein by reference, discloses a portable device with an external actuator, a case capable of being held in one hand and a digital display indicating peak force applied to the actuator. Strain gages connected in a bridge circuit are located on a flexure contacted by the actuator. The flexure is a cantilever beam shaped to fit the small case and to prevent reflected or mounting stresses being registered in the force display. While this device is capable of being used for AK, there is no provision for stable mounting, nor are there any provisions for substances being tested in conjunction with the individual.

### SUMMARY OF THE INVENTION

This invention resides in applied kinesiology (AK) testing apparatus and methods that eliminates uncertainties associated with applied kinesiology (AK) processes and provides consistent standardized results by accurately measuring a patient's resistance throughout an evaluation cycle. The patient applies a standard measured resistance, and when the item to be tested is held by the patient, the machine measures the decrease in resistance from the body, thereby accurately quantifying the patient's response to the food or other item being tested.

In terms of apparatus, the invention comprises a housing and a vertical member extending upwardly from the housing. The member includes a lower portion that enters into the housing wherein it is coupled to a device to measure applied pressure. The measurement device is in communication with a readout on the housing that indicates the applied pressure. The vertical member has an upper end coupled to a platform adapted to receive a pressure-producing body part, and a receptacle coupled to the platform is provided to hold a food or material that may be associated with an allergic reaction.

In one embodiment the device to measure applied pressure may be a mechanical weight scale. Alternatively, the device to measure applied pressure includes an electronic sensor in communication with a numerical readout. The vertical member may include a height adjustment mechanism. The platform is preferably in the form of a cradle to receive a person's wrist or arm. The system may further including tabs or lugs enabling the housing to be fastened to a stable surface.

A method of determining if a person is allergic to a food or other item, comprising the steps of:
placing the food or other item in the basket;
pressing down on the platform a predetermined number of times by a user, each time for a predetermined amount of time obtain a baseline pressure indication using the readout;
retrieving the food or other item from the receptacle;
repeating step of applying pressure while holding the food or other item; and
determining that the user is allergic to the food or other item if the average pressure indications of while holding the food or other item are statistically lower than the baseline pressure indication. In the preferred embodiment, the user applies pressure to the platform with a forearm or other portion of a user's aim at least twice for at least 10 seconds in each case. More preferably the user applies pressure three times for at least 40 seconds in each case.

BRIEF DESCRIPTION OF THE DRAWINGS

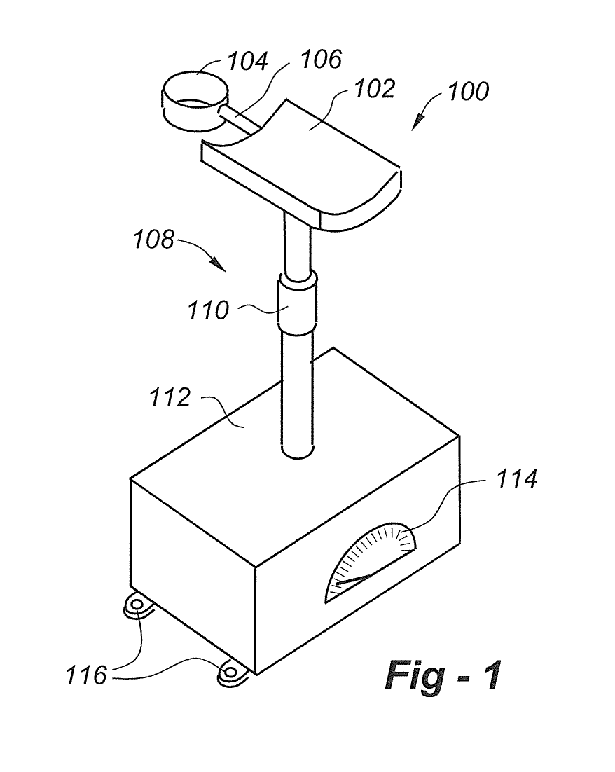

FIG. 1 is a drawing of the preferred apparatus of the invention.

### DETAILED DESCRIPTION OF THE INVENTION

This invention eliminates uncertainties associated with applied kinesiology (AK) processes and provides consistent standardized results by accurately measuring a patient's resistance throughout an evaluation. The patient applies a standard measured resistance, and when the item to be tested is held by the patient, the machine measures the decrease in resistance from the body, thereby accurately quantifying the patient's response to the food or other item being tested.

FIG. 1 is a drawing of the preferred embodiment of the invention depicted generally at 100. The apparatus comprises a housing 112 coupled to a cradle 102 adapted to receive a human body part such as a forearm. The cradle 102 is coupled to the housing through a vertical support 108 that may include a height adjustment ring 110. The cradle is further coupled to a cup or basket 104 through a link 106. The housing 112 may further include lugs 116 enabling the unit to be fastened to a support surface for enhanced stability.

In operation, when a user presses down on the cradle 102, the readout 114 displays the applied force in appropriate units such as pounds, kilograms, etc. The readout may be mechanical, as shown, in which case the cradle 102 and support 108 are coupled to the readout 114 in the same way as a weight-measurement scale. Alternatively, the vertical support 108 may interface with a flexure driving electronic circuitry to provide an active numerical readout. As one example, the mechanism and circuitry described in the '148 patent referenced may be adapted for such purpose.

The correct method of utilizing the machine 100 is for the patient to slide his/her wrist against the device of the top of the machine and push for a predetermined period of time in the range of 10-60 seconds, more preferably on the order of 40 seconds. The meter will indicate the force being utilized and should be done at least twice, more preferably three or more times. The average of the applied pressures in each case now provide a stabilized force that the testing will be based upon.

The patient then slides his/her arm forward and reaches into the basket 104 to pick up the food that the patient may be allergic to. The patient then slides his/her arm back to the original position to measure the force with the patient holding the suspected food product and repeats the test. If the patient is allergic to the food product he/she will not be able to sustain the force previously accomplished.

The machine can be utilized by using the patient's other arm to touch various organs. If for instance, a rib is out, the patient's finger can be applied to the various ribs and when the rib that is out of adjustment is touched, the patient will go weak. The same applies to the patient's diaphragm being out of adjustment, it can also be placed on the thyroid and should the patient have hypothyroidism the patient will go weak. If adrenals are depleted and the same process will occur if a finger is placed over the adrenals, the same applies to many organs of the body.

1. Applied kinesiology testing apparatus, comprising:
a housing;
a vertical member having a lower portion entering into the housing and coupled to a device to measure applied pressure;
a readout on the housing indicating the applied pressure;
the vertical member having an upper end coupled to a platform adapted to receive a pressure-producing body part; and
a receptacle coupled to the platform for hold a food or material associated with an allergic reaction.

2. The apparatus of claim 1, wherein the device to measure applied pressure is a mechanical weight scale.

3. The apparatus of claim 1, wherein the device to measure applied pressure includes an electronic sensor in communication with a numerical readout.

4. The apparatus of claim 1, wherein the vertical member includes a height adjustment mechanism.

5. The apparatus of claim 1, wherein the platform is a cradle to receive a person's wrist or arm.

6. The apparatus of claim 1, further including tabs or lugs enabling the housing to be fastened to a stable surface.

7. A method of determining if a person is allergic to a food or other item, comprising the steps of:
(a) providing the apparatus of claim 1;
(b) placing the food or other item in the basket;
(c) pressing down on the platform a predetermined number of times by a user, each time for a predetermined amount of time obtain a baseline pressure indication using the readout;
(d) retrieving the food or other item from the receptacle;
(e) repeating step (c) while holding the food or other item; and
(f) determining that the user is allergic to the food or other item if the average pressure indications of step (e) are statistically lower than those of step (c).

8. The method of claim 7, wherein the user applies pressure to the platform with a forearm or other portion of a user's arm.

9. The method of claim 7, wherein in steps (c) and (e), the user applies pressure at least twice for at least 10 seconds in each case.

10. The method of claim 7, wherein in steps (c) and (e), the user applies pressure three times for at least 40 seconds in each case.
